(19) United States
(12) Patent Application Publication
(10) Pub. No.: US 2014/0234240 A1
(43) Pub. Date: Aug. 21, 2014

(54) LESS YELLOWING NAIL COATING

(71) Applicant: O P I Products, Inc., (US)

(72) Inventor: Sunil J. Sirdesai, Irvine, CA (US)

(73) Assignee: O P I Products, Inc., North Hollywood, CA (US)

(21) Appl. No.: 13/773,457

(22) Filed: Feb. 21, 2013

(51) Int. Cl. A61K 8/55; A61Q 3/02; A61K 8/35; A61K 8/37; A61K 8/34

(52) U.S. Cl. CPC A61K 8/55; A61K 8/37; A61K 8/34; A61K 8/35; A61Q 3/02
USPC 424/61

(57) ABSTRACT

A composition for forming a less yellowing coating on a nail. The composition includes a curable resin, a monomer, a photoinitiator and a photosynergist comprising a methacrylate primary amine monomer. The photoinitiator may be a type I or a type II photoinitiator. The photosynergist may be dimethylamino ethyl methacrylate (DMAEM) or diethylaminoethyl methacrylate (DEAEM).

### BACKGROUND

1. Field

An embodiment of the invention is directed to a nail coating including a less yellowing photosynergist for use with photoinitiators. Other embodiments are also described and claimed.

2. Background

There are many nail formulations which rely upon exposure to light to activate (e.g., cure) the formulation (e.g., gel) on the nail. The formulation may therefore include photosynergists which are used to enhance the photodissociation of photoinitiators within the formulation when the formulation is exposed to ultra violet (UV) light. A photosynergist is a molecule with a primary amino end-group and is yellow in color, as is typical of amines. Almost all photosynergists used in nail formulations are simple compounds, acrylated, and polyethylene glycol based amines.

A photoinitiator is a chemical compound that decomposes into free radicals when exposed to light. Photoinitiators are added to a formulation to convert absorbed light energy, UV or visible light, into chemical energy in the form of initiating species, free radicals or cations. Based on the mechanism by which initiating radicals are formed, photoinitiators are generally divided into two classes: type I photoinitiators and type II photoinitiators. UV photoinitiators of both type I and type II are available, however, visible light photoinitiators belong almost exclusively to the type II class of photoinitiators. Photoinitiators are generally yellow in color, with the type II photoinitiators having the deepest yellow hue. In general, the deeper one goes into the visible light range, the yellower are the photoinitiators.

Since both photosynergists and photoinitiators may be yellow in color, although they provide several advantages to the formulations in which they are used, they also cause the formulation to take on a yellow hue. This yellow coloring is typically undesirable in nail formulations (e.g., nail polish, gel overlays, etc.) because it interferes with the desired appearance of the coating on the nail.

### SUMMARY

An embodiment of the invention is directed to a nail coating composition which includes a photoinitiator and a photosynergist which provide a nail coating composition having less yellowing than typical nail coatings including a photoinitiator and a photosynergist. Representatively, the composition is at least 2 scales lower on the ASTM D6166 Gardner Color Scale than typical nail coatings. The reduced yellowing is accomplished by combining a methacrylated amine photosynergist with a photoinitiator. For example, the methacrylated amine photosynergist may be dimethylamino ethyl methacrylate (DMAEM) or diethylaminoethyl methacrylate (DEAEM). The photoinitiator may be a photoinitiator which can be excited within an ultraviolet A wavelength range or a visible light range (e.g., a type II photoinitiator). It is believed that the yellow color of, for example, the UVA photoinitiator masks the yellowing property of the methacrylated amine photosynergist thus resulting in a less yellow composition, as compared to a formulation including a conventional photosynergist/UVA photoinitiator combination.

The above summary does not include an exhaustive list of all aspects of the embodiments disclosed herein. It is contemplated that the embodiments may include all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### DETAILED DESCRIPTION

In this section we shall explain several preferred embodiments of the invention. Whenever the ingredients, ranges and other aspects of the formulations described in the embodiments are not clearly defined, the scope of the embodiments is not limited only to the formulations shown, which are meant merely for the purpose of illustration. Also, while numerous details are set forth, it is understood that some embodiments may be practiced without these details. In other instances, well-known structures and techniques have not been shown in detail so as not to obscure the understanding of this description.

The instant invention is directed to a composition designed to provide overlays (e.g., a nail coating) on the nail of a user. In one embodiment, the composition may be a composition which, when applied to the nail, forms a gel coating over the nail. The composition may be a photoreactive composition in that it cures (i.e., forms a gel coating) when exposed to a light source. In this aspect, the composition may include a photosynergist and a photoinitiator. As previously discussed, photosynergists and photoinitiators have a yellow color which can cause formulations in which they are used to also take on a yellow hue. It was unexpectedly found, however, that a nail coating composition including a methacrylate amine photosynergist and a photoinitiator, such as a type I photoinitiator or a type II photoinitiator, for example, that cures under UVA light or visible light, experiences less yellowing than other formulations using amine photosynergists and a UVA photoinitiator. Representatively, the composition experiences less yellowing by at least 2 values on the ASTM D6166 Gardner Color Scale than other formulations using amine photosynergists and a UVA photoinitiator. For example, a typical formulation may have an ASTM D6166 Gardner Color Scale value of from about 7 to 8, whereas the nail coating composition disclosed herein has a value of less than 7, for example, from 6 to 4 on the ASTM D6166 Gardner Color Scale. Such a result is unexpected because the deeper one goes into the UVA or visible light photoinitiator range, the yellower the photoinitiators become. It would therefore not be expected that yellowing of a formulation could be reduced by combining a photosynergist having a yellow hue with a photoinitiator having an even yellower hue. Rather, one would expect that the composition would maintain its yellow appearance or be even yellower, regardless of the photosynergist used. It was unexpectedly found, however, that the composition described herein is actually less yellow because the methacrylated amine photosynergist forms an integral part of polymer (chemical bonding) and cannot rise to the surface.

Thus, in some embodiments, the composition may include a methacrylated amine photosynergist. Suitable methacrylated amine photosynergists may include, but are not limited to, one or more of, dimethylamino ethyl methacrylate (DMEAM) and diethylamino ethyl methacrylate (DEAEM). It is noted that DMAEM and DEAEM cure to become an integral part of the nail coating and therefore do not suffer from the disadvantages of other small molecule amine photosynergists which migrate to the coating surface thereby further enhancing the yellowness of the coating. In one embodiment, the composition may include the photosynergist in any amount sufficient to form a less yellowing coating (e.g., a gel overlay) on a user's nail. For example, in one embodiment, the composition may include the photosynergist in an amount of from about 0.4 percent to about 1 percent, for example, about 0.5 percent by weight of the total composition.

Suitable photoinitiators that can be used with the above described photosynergists to provide a less yellow composition may be type I photoinitiators or type II photoinitiators. In some cases, the photoinitiator is one which is excitable when exposed to radiation within a UVA wavelength range (e.g., 400-315 nm) or, in some cases, a visible light range (e.g., 380-750 nm). Representative type I photoinitiators may include, but are not limited to, trimethylbenzoyl diphosphine oxide, trimethylbenzoyl diphenylphosphine oxide, or other phosphine oxides and their derivatives, benzoyl isopropanol, hydroxycyclohexyl phenyl ketone, or ethyl trimethylbenzoylphenylphosphinate. Representative type II photoinitiators may include, but are not limited to, xanthones, camphorquinones and the like. In some embodiments, the composition may include two or more different photoinitiators which are excited at different wavelength ranges. For example, in one embodiment, the composition may include a photoinitiator which can be excited in the 365 nm region and a photoinitiator which can be excited in the 405 nm region. Representatively, the composition may include an alpha-hydroxy ketone photoinitiator (e.g., benzoyl isopropanol or hydroxycyclohexyl phenyl ketone) and a phosphine oxide photoinitiator (e.g., trimethylbenzoyl diphosphine oxide, trimethylbenzoyl diphenylphosphine oxide). The composition may include one or more of a photoinitiator in any amount sufficient to facilitate formation of a gel overlay on a user's nail. For example, in one embodiment, the composition may include the photoinitiator(s) in an amount of from about 0.5 percent to about 5.5 percent, for example, from about 1 percent to about 4.7 percent, or from about 3.4 percent to about 4.2 percent.

The composition may further include a curable resin and a monomer to facilitate formation of the coating upon application of the composition to the nail. The curable resin may be, but is not limited to, urethane acrylate/methacrylate oligomers, for example, urethane (meth)acrylate. In one embodiment, the composition may include the curable resin in any amount sufficient to form a coating on a user's nail. For example, in one embodiment, the composition may include the curable resin in an amount of from about 35 percent to about 65 percent, for example, from about 39 percent to about 58.4 percent, or from 49 percent to 55 percent by weight of the total composition.

The monomer may be any type of monomer suitable for use within a composition for forming a nail coating. In some cases, the monomer may also serve as a crosslinker within the gel system. For example, in one embodiment, the monomer may be, but is not limited to, one or more of triethylene glycol dimethacrylate, hydroxypropyl methacrylate, isobornyl methacrylate, isobornyl acrylate, hydroxyethyl methacrylate (HEMA), hema maleate, PEG-4 dimethacrylate or ethyl methacrylate. The composition may include the monomer in any amount sufficient to facilitate formation of a coating on a user's nail. For example, in one embodiment, the composition may include the monomer(s) in an amount of from about 25 percent to about 60 percent, for example, from about 30.6 percent to about 55.3 percent, or from 36 percent to 40.8 percent by weight of the total composition.

In some embodiments, in addition to the photosynergist(s), photoinitiator(s), curable resin(s) and monomer(s), the base component may include a thixotrope(s), a solvent(s), a plasticizer(s), a pigment(s) and/or colorant(s). A suitable thixotrope may include, but is not limited to, polyquarternium 37. The thixotrope may be provided in any amount sufficient to facilitate formation of a coating on a user's nail, for example, from about 2 percent to about 10 percent, or from about 4 percent to 6 percent, or at least 5 percent by weight of the total composition.

A suitable solvent may include, but is not limited to, propylene glycol dicaprylate. The solvent may be provided in any amount sufficient to facilitate formation of a coating on a user's nail, for example, from about 0.5 percent to about 10 percent, or from about 1 percent to 5 percent, or at least 2 percent by weight of the total composition.

A suitable plasticizer may include, but is not limited to, PPG-1 trideceth 6 or dimethicone. The plasticizer may be provided in any amount sufficient to facilitate formation of a coating on a user's nail, for example, from about 0.5 percent to about 10 percent, or from about 1 percent to 5 percent, or at least 2 percent by weight of the total composition.

Suitable pigments may include, but are not limited to, titanium dioxide, iron oxide or red 21, in any amount sufficient to achieve a coating of the desired appearance. The pigment may be provided in any amount sufficient to enhance an appearance of the composition and/or provide the aesthetic characteristics desired. For example, in one embodiment, the composition may include a pigment in an amount of from about 0.5 percent to about 5 percent, for example, from 1 percent to 3 percent, representatively, from 1.5 percent to 2.5 percent by weight of the total composition.

Suitable colorants may include, but are not limited to, violet 2, red 30 or any other colorant in any amount sufficient to achieve a coating of the desired appearance. The colorant may be provided in any amount sufficient to enhance an appearance of the composition and/or provide the aesthetic characteristics desired. For example, in one embodiment, the composition may include a colorant in an amount of from about 0.1 percent to about 5 percent, for example, from 0.2 percent to 3 percent, representatively, about 0.3 percent by weight of the total composition.

The composition may further include an additive in any amount sufficient to enhance an appearance of the composition and/or provide the aesthetic characteristics desired. For example, in one embodiment, the composition may include an additive such as glitter in an amount of from about 0.5 percent to about 5 percent, or from about 2.5 percent to about 3.5 percent, representatively, 3 percent by weight of the total composition.

Exemplary nail coating formulations are provided below.

### Example 1

| | | Weight |
|---|---|---|
| | | Percent |
| Chemical Name | Function | (%) |
| Urethane Acrylate/Methacrylate | Curable Resin | 56.5 |
| Oligomers | | |
| Triethylene Glycol Dimethacrylate | Monomer/Crossslinker | 27.1 |
| Benzoyl Isopropanol | Photoinitiator | 5 |
| Trimethylbenzoyl Diphosphine Oxide | Photoinitiator | 0.4 |
| Polyquaternium 37 | Thixotrope | 5 |
| Propylene Glycol Dicaprylate | Solvent | 2 |
| PPG-1 Trideceth 6 | Plasticizer | 2 |
| Titanium Dioxide | Pigment | 1 |
| Dimethylamino ethyl methacrylate | Monomer/Photosynergist | 1 |
| (DMAEM) | | |

### Example 2

| | | Weight |
|---|---|---|
| | | Percent |
| Chemical Name | Function | (%) |
| Urethane Acrylate/Methacrylate | Curable Resin | 52.5 |
| Oligomers | | |
| Hydroxypropyl Methacrylate | Monomer | 14 |
| Isobornyl Methacrylate | Monomer | 8.6 |
| HEMA | Monomer | 8 |
| Triethylene Glycol Dimethacrylate | Monomer/Crosslinker | 7 |
| Hydroxycyclohexyl Phenyl Ketone | Photoinitiator | 5 |
| DMAEM | Monomer/Photosynergist | 1.0 |
| Ethyl | Photoinitiator | 0.4 |
| Trimethylbenzoylphenylphosphinate | | |
| Glitter | Additive | 3.5 |

### Example 3

| | | Weight |
|---|---|---|
| | | Percent |
| Chemical Name | Function | (%) |
| Urethane Acrylate/Methacrylate Oligomers | Curable Resin | 57.5 |
| Triethyleneglycol Dimethacrylate | Monomer/ | 27 |
| | Crosslinker | |
| Diethylaminoethyl Methacrylate (DEAEM) | Monomer/ | 1 |
| | Photosynergist | |
| HEMA | Monomer | 8.6 |
| Hydroxycyclohexyl Phenyl Ketone | Photoinitiator | 3 |
| Ethyl Trimethylbenzoylphenylphosphinate | Photoinitiator | 0.4 |
| Glitter | Additive | 2.5 |

### Example 4

| | | Weight |
|---|---|---|
| | | Percent |
| Chemical Name | Function | (%) |
| Urethane Acrylate/Methacrylate Oligomers | Curable Resin | 39 |
| Hydroxypropyl Methacrylate | Monomer | 26.5 |
| Isobornyl Methacrylate | Monomer | 23.8 |
| DMAEM | Monomer/ | 0.5 |
| | Photosynergist | |
| Henna Maleate | Monomer | 5 |
| Benzoyl Isopropanol | Photoinitiator | 5 |
| Ethyl Trimethylbenzoylphenylphosphinate | Photoinitiator | 0.2 |

### Example 5

| | | Weight |
|---|---|---|
| | | Percent |
| Chemical Name | Function | (%) |
| Urethane Acrylate/Methacrylate Oligomers | Curable Resin | 58.4 |
| PEG-4 Dimethacrylate | Monomer/ | 21 |
| | Crosslinker | |
| Ethyl Methacrylate | Monomer | 10 |
| Isobornyl Acrylate | Monomer | 5 |
| Trimethylbenzoyl Diphenylphosphine Oxide | Photoinitiator | 1 |
| DMAEM | Monomer/ | 0.4 |
| | Photosynergist | |
| Violet 2 | Colorant | 0.2 |

### Example 6

| | | Weight |
|---|---|---|
| | | Percent |
| Chemical Name | Function | (%) |
| Urethane Acrylate/Methacrylate Oligomers | Curable Resin | 55 |
| Triethylene Glycol Dimethacrylate | Monomer/ | 30.5 |
| | Crosslinker | |
| DMAEM | Monomer/ | 0.5 |
| | Photosynergist | |
| HEMA | Monomer | 6 |
| Hydroxycyclohexyl Phenyl Ketone | Photoinitiator | 2.5 |
| Benzoyl Isopropanol | Photoinitiator | 2.5 |
| Ethyl Trimethylbenzoylphenylphosphinate | Photoinitiator | 0.2 |
| Red 30 | Colorant | 0.3 |
| Glitter | Additive | 2.5 |

### Example 7

| | | Weight |
|---|---|---|
| | | Percent |
| Chemical Name | Function | (%) |
| Urethane Acrylate/Methacrylate Oligomers | Curable Resin | 50.5 |
| Triethylene Glycol Dimethacrylate | Monomer/ | 28 |
| | Crosslinker | |
| Isobornyl Acrylate | Monomer | 9 |
| HEMA | Monomer | 3.8 |
| DEAEM | Monomer/ | 0.5 |
| | Photosynergist | |
| Hydroxycyclohexyl Phenyl Ketone | Photoinitiator | 4 |
| Trimethylbenzoyl Diphenylphosphine Oxide | Photoinitiator | 0.2 |
| Isobornyl Methacrylate | Monomer | 1 |
| Glitters | Additives | 3 |

### Example 8

| | | Weight |
|---|---|---|
| | | Percent |
| Chemical Name | Function | (%) |
| Urethane Acrylate/Methacrylate Oligomers | Curable Resin | 52 |
| HEMA | Monomer | 20.6 |
| Isobornyl Acrylate | Monomer | 12 |
| Hydroxycyclohexyl Phenyl Ketone | Photoinitiator | 2 |
| Benzoyl Isopropanol | Photoinitiator | 2 |
| Ethyl Trimethylbenzoylphenylphosphinate | Photoinitiator | 0.4 |
| Dimethicone | Plasticizer | 1 |
| Isobornyl Methacrylate | Monomer | 4 |
| Glitters | Additives | 5 |
| DMAEM | Monomer/ | 1 |
| | Photosynergist | |

### Example 9

| | | Weight |
|---|---|---|
| | | Percent |
| Chemical Name | Function | (%) |
| Urethane Acrylate/Methacrylate Oligomers | Curable Resin | 49 |
| Triethylene Glycol Dimethacrylate | Monomer/ | 27.2 |
| | Crosslinker | |
| DMAEM | Monomer/ | 1 |
| | Photosynergist | |
| HEMA | Monomer | 11.9 |
| Benzoyl Isopropanol | Photoinitiator | 2 |
| Hydroxycyclohexyl Phenyl Ketone | Photoinitiator | 2 |
| Ethyl Trimethylbenzoylphenylphosphinate | Photoinitiator | 0.4 |
| Titanium Dioxide | Pigment | 1 |
| Iron Oxide | Pigment | 1 |
| Red 21 | Pigment | 1 |
| Glitters | Additive | 3.5 |

### Example 10

| | | Weight |
|---|---|---|
| | | Percent |
| Chemical Name | Function | (%) |
| Urethane Acrylate/Methacrylate Oligomers | Curable Resin | 61.5 |
| Hydroxypropyl Methacrylate | Monomer | 17.5 |
| HEMA | Monomer | 15.3 |
| Hydroxycyclohexyl Phenyl Ketone | Photoinitiator | 4.5 |
| Trimethylbenzoyl Diphenylphosphine Oxide | Photinitiator | 0.2 |
| DMAEM | Monomer/ | 0.5 |
| | Photosynergist | |
| Glitters | Additive | 0.5 |

A composition suitable for forming a coating on a nail including any of the foregoing formulations may be formed according to any suitable technique. Representatively, in one embodiment, a curable resin(s), a monomer(s), a photoinitiator(s), photosynergist(s), a thixotrope(s), a solvent(s), a plasticizer(s), a pigment(s) and/or colorant(s) may be mixed to form the composition. Alternatively, the composition may be formed by mixing any one or more of the ingredients of any of the above-referenced exemplary formulations together in any order and in any combination.

In the foregoing specification, the invention has been described with reference to specific embodiments thereof. It will, however, be evident that various modifications and changes can be made thereto without departing from the broader spirit and scope of the invention as set forth in the appended claims. For example, although a nail coating such as a gel coating is disclosed herein, it is contemplated that the composition may form any type of curable covering for a nail, for example, an artificial nail, a top-coat which is applied over a nail lacquer, or the like. The specification is, accordingly, to be regarded in an illustrative rather than a restrictive sense.

What is claimed is:

1. A composition for forming a less yellowing coating on a nail comprising:
a curable resin;
a monomer;
a photoinitiator; and
a photosynergist comprising a methacrylate primary amine monomer, and wherein the composition forms a coating with a less yellow appearance.

2. The composition of claim 1 wherein the photosynergist is one of dimethylamino ethyl methacrylate (DMAEM) or diethylaminoethyl methacrylate (DEAEM).

3. The composition of claim 1 wherein the photosynergist is in an amount of from 0.4 percent to 1 percent by weight of the total composition.

4. The composition of claim 1 wherein the photoinitiator is a phosphine oxide or derivatives thereof.

5. The composition of claim 1 wherein the photoinitiator is a type I photoinitiator selected from the group consisting of benzoyl isopropanol, trimethylbenzoyl diphosphine oxide, trimethylbenzoyl diphenylphosphine oxide, hydroxycyclohexyl phenyl ketone, and ethyl trimethylbenzoylphenylphosphinate.

6. The composition of claim 1 wherein the photoinitiator is a type II photoinitiator selected from one of a xanthone or a camphorquinone.

7. The composition of claim 1 wherein the photoinitiator is in an amount of from 0.5 percent to 5.5 percent by weight of the total composition.

8. The composition of claim 1 wherein the curable resin comprises urethane acrylate or methacrylate oligomers.

9. The composition of claim 1 wherein the curable resin is in an amount of from 35 percent to 65 percent by weight of the total composition.

10. The composition of claim 1 wherein the monomer is selected from the group consisting of triethylene glycol dimethacrylate, hydroxypropyl methacrylate, isobornyl methacrylate, isobornyl acrylate, hydroxyethyl methacrylate (HEMA), hema maleate, PEG-4 dimethacrylate and ethyl methacrylate.

11. The composition of claim 1 wherein the monomer is in an amount of from 25 percent to 60 percent by weight of the total composition.

12. The composition of claim 1 wherein the coating forms a gel overlay which covers the entire nail.

13. The composition of claim 1 wherein the composition is less yellow by being least 2 values lower on the ASTM D6166 color scale than another nail coating including a photoinitiator and a different photosynergist.

14. A photoreactive composition for forming a less yellowing coating on a nail comprising:
a curable resin in an amount of from 35 percent to 65 percent by weight of the total composition;
a monomer in an amount of from 25 percent to 60 percent by weight of the total composition;
a photoinitiator in an amount of from 0.5 percent to 5.5 percent by weight of the total composition; and
a photosynergist comprising a methacrylate primary amine monomer in an amount of one percent or less by weight of the total composition, and
wherein the composition forms a coating with a less yellow appearance.

15. The photoreactive composition of claim 14 wherein the photoinitiator is a first photoinitiator, the composition further comprising a second photoinitiator, and wherein the second photoinitiator is excitable within a different wavelength range than the first photoinitiator.

16. The photoreactive composition of claim 14 wherein the photoinitiator is a type II photoinitiator.

17. The photoreactive composition of claim 14 wherein the photosynergist is one of dimethylamino ethyl methacrylate (DMAEM) or diethylamino ethyl methacrylate (DEAEM).

18. The photoreactive composition of claim 14 wherein the photosynergist is in an amount of from 0.4 percent to 1 percent by weight of the total composition.

19. The photoreactive composition of claim 14 further comprising:
a thixotrope in an amount of at least 5 percent by weight of the total composition; and
a solvent in an amount of at least 2 percent by weight of the total composition.

20. A composition for forming a less yellowing coating on a nail comprising:
a curable resin in an amount of from 35 percent to 65 percent by weight of the total composition;
a monomer in an amount of from 25 percent to 60 percent by weight of the total composition;
a photoinitiator in an amount of from 0.5 percent to 5.5 percent by weight of the total composition, wherein the photoinitiator is excitable within an ultraviolet A wavelength (UVA) range; and
a photosynergist in an amount of one percent or less by weight of the total composition, wherein the photosynergist comprises a methacrylate primary amine monomer, and
wherein the composition forms a coating with a less yellow appearance.

21. The composition of claim 20 wherein the photosynergist is one of dimethylamino ethyl methacrylate (DMAEM) or diethylaminoethyl methacrylate (DEAEM).

22. The composition of claim 20 wherein the composition is less yellow in that it has an ASTM D6166 Gardner Color Scale value of less than 7.
